SEQUENCE LISTING (3 sequences)

SEQ ID NO: 1 - protein, 47 aa, synthetic construct
MESRGRRCPEMISVLGPISGHVLKAVFSRGDTPVLPHETRLLQTGIH

SEQ ID NO: 2 - protein, 38 aa, synthetic construct
AVIHASGKQMWQARLTVSGLAWTRQQNQWKEPDVYYTS

SEQ ID NO: 3 - protein, 33 aa, synthetic construct
AIRETVELRQYDPVAALFFFDIDLLLQRGPQYS

(19) United States
(12) Patent Application Publication
(10) Pub. No.: US 2025/0314653 A1
(43) Pub. Date: Oct. 9, 2025

(54) DETECTION OF ANTI-VIRAL CDR3s IN NEUROBLASTOMA

(71) Applicant: University of South Florida, Tampa, FL (US)

(72) Inventors: George Blanck, Tampa, FL (US); Dorottya Kacsoh, Orlando, FL (US); Andrea Chobrutskiy, Portland, OR (US); Boris Chobrutskiy, Portland, OR (US)

(21) Appl. No.: 18/921,935

(22) Filed: Oct. 21, 2024

Related U.S. Application Data
(60) Provisional application No. 63/631,541, filed on Apr. 9, 2024.

(51) Int. Cl. G01N 33/574; G01N 33/569

(52) U.S. Cl. CPC G01N 33/57407; G01N 33/56972; G01N 33/56983; G01N 2333/045; G01N 2469/20

(57) ABSTRACT

The present disclosure relates to methods of determining complementarity scores and treatment based on the association between the blood-based T-cell receptor anti-viral CDR3s and viral antigens associated with worse overall survival for neuroblastoma subjects.

### CROSS REFERENCE TO RELATED APPLICATIONS

This application claims the benefit of U.S. Provisional Patent Application Ser. No. 63/631,541, filed on Apr. 9, 2024, the disclosure of which is expressly incorporated herein by reference in its entirety.

### REFERENCE TO SEQUENCE LISTING

The sequence listing submitted on Dec. 29, 2024, as an .XML file entitled “11001-195US1_ST26.xml” created on Oct. 23, 2024, and having a file size of 3,763 bytes is hereby incorporated by reference pursuant to 37 CFR § 1.832.

### FIELD

The present disclosure relates to methods of determining complementarity score of blood-based CDR3 sequences and viral antigens and predicting overall survival probabilities in neuroblastoma subjects. The scoring is based on TCR CDR3 sequence complementarity in immune responses against viral infections and their potential role in cancer immunotherapy.

### BACKGROUND

Neuroblastoma (NBL) is a childhood cancer originating from the sympathetic nervous system, specifically from cells of the sympathetic chain. This malignancy is known for its heterogeneous nature, displaying a wide range of clinical behaviors and outcomes. Over the past several decades, researchers have observed that many patients diagnosed with primary neuroblastoma tumors are seropositive for cytomegalovirus (CMV) antibodies before the initiation of any treatment. This seropositivity suggests a potential association between CMV infection and the development or progression of neuroblastoma.

The presence of CMV in various cancers might contribute to immune evasion by tumor cells, thereby promoting an immunosuppressive tumor microenvironment. CMV infection in tumor cells is often linked to elevated expression of the MYCN gene, a known marker of poor prognosis in neuroblastoma. With the advent of large collections of adaptive immune receptor recombination reads representing cancer, there is the opportunity to further investigate the adaptive immune response to viruses in the cancer setting. This is a particularly important goal due to longstanding but still not well-resolved questions about viral etiologies in cancer and viral infections as comorbidities.

Overall, there is a significant need for a novel strategy for assessing viral infection complications in NBL patients and thereby providing effective adaptive immune responses. Therefore, what is needed are new methods for diagnosing and treating cancer patients. The methods disclosed herein address these and other needs.

### SUMMARY

Disclosed herein are methods for determining survival outcome of a subject with neuroblastoma based on complementarity scoring. Determining the complementarity scores based on T-cell receptor anti-viral CDR3s and proteins associated with cancers and uses thereof in treating, diagnosing, and/or prognosing cancers.

In some examples, disclosed herein is a method of determining overall survival probabilities in a subject with neuroblastoma, comprising:
a) obtaining a blood sample from the subject, wherein the blood sample comprises a T-cell receptor (TCR);
b) extracting complementarity determining region 3 (CDR3) amino acid (AA) sequences from the T-cell receptor (TCR) in the blood sample, thereby obtaining extracted CDR3 AA sequences;
c) identifying an exact match of extracted CDR3 AA sequences to known anti-viral CDR3 AA sequences from the blood sample; thereby obtaining an exact match anti-viral CDR3 AA sequence; and
d) correlating presence of the exact match anti-viral CDR3 AA sequence with overall survival probability of the subject,

wherein the presence of the exact match anti-viral CDR3 AA sequence in the blood sample is correlated to poor overall survival compared to a reference control, wherein the reference control does not have the exact match anti-viral CDR3 AA sequence in the blood sample.

In some examples, the known anti-viral CDR3 AA sequences of one or more viruses selected from the group consisting of Cytomegalovirus (CMV), Epstein-Barr virus (EBV), Hepatitis C virus (HCV), human immunodeficiency virus (HIV), Influenza A, and SARS-CoV-2.

In some examples, the T cell receptor (TCR) comprises an alpha chain or a beta chain.

In some examples, disclosed herein is a method of determining overall survival in a subject with neuroblastoma, comprising:
a) obtaining a sample from the subject, wherein the sample comprises a T-cell receptor (TCR);
b) extracting complementarity determining region 3 (CDR3) from the T-cell receptor (TCR) in the sample, thereby obtaining extracted CDR3;
c) obtaining a chemical complementarity score (CS) by interacting known viral antigens to the extracted CDR3 from the sample;
d) calculating the CS; and
e) correlating the CS with overall survival of the subject,

wherein a high CS is correlated to poor overall survival compared to a reference control, wherein the reference control has low CS.

In some examples, the high CS comprises a high chemical complementarity between the known viral antigens and the extracted CDR3.

In some examples, the CS is calculated using hydrophobic interactions, electrostatic interactions, or a combination thereof.

In some examples, the sample comprises blood or tumor biopsy.

In some examples, the known viral antigens of one or more viruses selected from the group consisting of Cytomegalovirus (CMV), Epstein-Barr virus (EBV), Hepatitis C virus (HCV), human immunodeficiency virus (HIV), Influenza A, and SARS-CoV-2.

In some examples, disclosed herein is a method of treating neuroblastoma in a subject, comprising:
a) obtaining a sample from the subject, wherein the sample comprises a T-cell receptor (TCR);
b) extracting complementarity determining region 3 (CDR3) amino acid (AA) sequences from the T-cell receptor (TCR) in the sample, thereby obtaining extracted CDR3 AA sequences;
c) identifying an exact match of extracted CDR3 AA sequences to anti-Cytomegalovirus (CMV) CDR3 AA sequences from the sample; thereby obtaining an exact match anti-CMV CDR3 AA sequence;
d) correlating presence of the exact match anti-CMV CDR3 AA sequence with overall survival of the subject; and
e) administering a therapeutically effective amount of an anti-CMV comprising Ganciclovir or Valganciclovir to the subject with a presence of the exact match anti-CMV CDR3 sequence,

wherein the presence of the exact match anti-CMV CDR3 AA sequence in the sample is correlated to poor overall survival.

In some examples, disclosed herein is a method of treating neuroblastoma in a subject, comprising:
a) obtaining a sample from the subject, wherein the sample comprises a T-cell receptor (TCR);
b) extracting complementarity determining region 3 (CDR3) from the T-cell receptor (TCR) in the sample, thereby obtaining extracted CDR3;
c) obtaining a chemical complementarity score (CS) by interacting CMV antigen to the extracted CDR3 from the sample;
d) calculating the CS;
e) correlating the CS with overall survival of the subject; and
f) administering a therapeutically effective amount of an anti-CMV comprising Ganciclovir or Valganciclovir to the subject with a high CS, wherein a high CS is correlated to poor overall survival.

In some examples, disclosed herein is a method of treating neuroblastoma in a subject based on correlation between chemical complementary scoring and gene amplification, comprising:
a) obtaining a sample from the subject, wherein the sample comprises a T-cell receptor (TCR);
b) extracting complementarity determining region 3 (CDR3) from the T-cell receptor (TCR) in the sample, thereby obtaining extracted CDR3;
c) obtaining a chemical complementarity score (CS) by interacting known viral antigen to the extracted CDR3 from the sample;
d) calculating the CS;
e) correlating the CS with overall survival of the subject; wherein a high CS is correlated to poor overall survival compared to a reference control, wherein the reference control has low CS;
f) determining gene amplification status of MYCN gene in the sample;
g) classifying the subject into a high-risk group based on the high CS and MYCN gene amplification in the sample; and
h) administering a therapeutically effective amount of an anti-viral comprising Ganciclovir or Valganciclovir to the high-risk group.

In some examples, disclosed herein is a method of treating neuroblastoma in a subject based on correlation between exact match sequencing and gene amplification, comprising:
a) obtaining a sample from the subject, wherein the sample comprises a T-cell receptor (TCR);
b) extracting complementarity determining region 3 (CDR3) amino acid (AA) sequences from the T-cell receptor (TCR) in the sample, thereby obtaining extracted CDR3 AA sequences;
c) identifying an exact match of extracted CDR3 AA sequences to known anti-viral CDR3 AA sequences from the sample; thereby obtaining an exact match anti-viral CDR3 AA sequence;
d) correlating presence of the exact match anti-viral CDR3 AA sequence with overall survival of the subject, wherein the presence of the exact match anti-viral CDR3 AA sequence in the sample is correlated to poor overall survival;
e) determining gene amplification status of MYCN gene in the sample;
f) classifying the subject into a high-risk group based on the presence of the exact match anti-viral CDR3 AA sequence and MYCN gene amplification in the sample; and
g) administering a therapeutically effective amount of an anti-viral comprising Ganciclovir or Valganciclovir to the high-risk group.

In some examples, disclosed herein is a kit for use in a method of detecting the presence of anti-viral T-cell receptors in a subject with cancer, comprising:
a) isolating a nucleic acid from a tumor sample derived from the subject;
b) sequencing a polynucleotide encoding a complementarity determining region (CDR) sequence of a T-cell receptor in the tumor microenvironment, thereby obtaining extracted CDR3 sequences;
c) identifying exact match anti-viral CDR3 sequences from the extracted CDR3 sequences, thereby obtaining the exact match anti-viral CDR3 sequences;
d) determining exact match anti-viral CDR3 sequences to known viral antigen sequences;
e) determining gene amplification status of MYCN gene in tumor cells of the subject; and
f) classifying the subject into a high-risk group using multivariate analysis based on the presence of both exact match anti-viral CDR3 sequences and MYCN gene amplification in the tumor cells.

In some examples, the CDR domain is a CDR3 domain.

In some examples, the cancer is selected from the group consisting of neuroblastoma, low-grade glioma, stomach adenocarcinoma, esophageal cancer, melanoma, lung squamous cell carcinoma, lung adenocarcinoma, breast cancer, cervical squamous cell carcinoma, bladder cancer, muscle invasive bladder cancer, and soft tissue sarcoma.

In some examples, the protein associated with the cancer is selected from the group consisting of MYC proto-oncogene neuroblastoma derived (MYCN), isocitrate dehydrogenase 1 (IDH1), Phosphatidylinositol-4,5-Bisphosphate 3-Kinase Catalytic Subunit Alpha (PIK3CA), B-Raf proto-oncogene (BRAF), Dynein heavy chain 9 (DNAH9), myosin heavy chain 1 (MYH1), Tenascin-R (TNR), Teneurin-1 (TNM1), Plexin-A4 (PLXNA4A), Microtubule-actin cross-linking factor 1 (MACF1), Tumor protein p53 (TP53), ATP-dependent helicase ATRX (ATRX), Neuroblastoma RAS viral oncogene homolog (NRAS), and Retinoblastoma protein (RB1).

### BRIEF DESCRIPTION OF THE DRAWINGS

The accompanying figures, which are incorporated in and constitute a part of this specification, illustrate several examples described below.

FIGS. 1A and 1B show Kaplan-Meier (KM) overall survival (OS) analysis representing case IDs with or without anti-CMV CDR3s and with or without MYCN amplification. FIG. 1A shows case IDs without MYCN amplification and with or without anti-CMV CDR3s; median survival of seropositive cases (n=53) (black), 32 months; median survival of seronegative cases (n=79) (grey), 82 months. Log-rank p-value, 1.074e-4. N-proportion test p-value=0.0001 at 154 months. FIG. 1B shows case IDs with MYCN amplification and with or without anti-viral CDR3s; median survival of seropositive cases (n=28) (black), 18 months; median survival of seronegative (n=36) (grey), 43 months. Log-rank p-value, 0.0576. N-proportion test p-value, 0.0721 at 63 months.

FIG. 2 shows KM OS analysis of case IDs positive for anti-CMV CDR3s and MYCN amplification. Case IDs positive for anti-CMV CDR3s and MYCN amplification (black, n=28; median survival, 18 months); case IDs negative for anti-CMV CDR3s and without MYCN amplification (grey, n=79; median survival, 82 months). Log-rank p-value=9.804e-5. N-proportion test p-value=0.0004 at 40 months.

FIGS. 3A, 3B and 3C show KM OS analyses representing case IDs grouped into upper and lower 50^{th} percentile groups for TRB CDR3-CMV pp65 peptide fragment CSs. FIG. 3A shows case IDs representing the upper 50^{th} percentile of CSs for blood-sourced, TRB CDR3s and pp65 fragment:

(SEQ ID NO: 1)
MESRGRRCPEMISVLGPISGHVLKAVFSRGDTPVLPHETRLLQTGIH

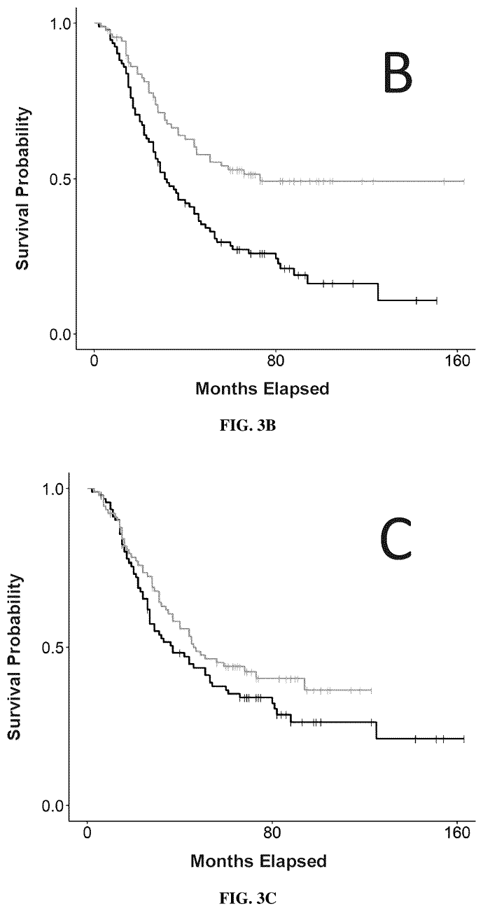

combinations (black, n=105; median survival, 31 months) and case IDs representing the lower 50^{th} percentile of the CSs (grey, n=75; median survival, NA). FIG. 3B shows case IDs representing the upper 50^{th} percentile of CSs for blood-sourced, TRB CDR3s and pp65 fragment AVIHASGKQMWQARLTVSGLAWTRQQNQWKEPDVYYTS (SEQ ID NO:2) combinations (black, n=92; median survival, 31 months) and case IDs representing the lower 50^{th} percentile of the CSs (grey, n=88; median survival, 73 months). FIG. 3C shows case IDs representing the upper 50th percentile of CSs for blood-sourced, TRB CDR3s and pp65 fragment AIRETVELRQYDPVAALFFFDIDLLLQRGPQYS (SEQ ID NO:3) combinations (black, n=91; median survival, 37 months) and case IDs representing the lower 50th percentile of the CSs (grey, n=89; median survival, 46 months).

### DETAILED DESCRIPTION

Neuroblastoma (NBL) is a childhood cancer that develops along part of the sympathetic nervous system called the sympathetic chain and has a variable prognosis based on various factors. For five decades or more, it has been known that patients with primary NBL tumors are often seropositive for cytomegalovirus (CMV) antibodies prior to beginning treatment. In fact, many different types of cancers have been found to be positive for CMV, and it has been hypothesized that CMV causes immune evasion of tumor cells and perpetuates an immunosuppressive tumor microenvironment. More specifically, it has been shown that the presence of CMV in primary tumor cells in NBL is oncogenic and that the longer it is present in tumor cells, the more malignant these tumor cells become. Frequently, tumor cells that have been persistently infected with CMV have an increased expression of MYCN. More recently, studies have shown that anti-viral treatment, such as ganciclovir, in NBL may offer survival benefits.

Adaptive immune receptors (IRs) have a hypervariable complementarity determining region-3 (CDR3) representing the amino acid (AA) sequence spanning the somatically occurring, recombination joining of the IR V- and J-gene segments. This CDR3 is highly important for antigen binding, and because of the known association between CMV and NBL, we investigated the relationship between the presence of anti-viral CDR3s of T-cell receptors (TCRs) in the blood and overall survival (OS). In addition to CMV, we also determined survival distinctions based on the presence of CDR3s known to bind antigens of other viruses. Because of the association between CMV and MYCN expression, the independent correlations of blood-based anti-CMV CDR3s and MYCN amplification with survival distinctions were assessed. Disclosed herein are the examples strongly indicating TCR AA sequences sourced from blood exome files and their usefulness in prognosis, or patient survival rates in tumors related to viral infections. The anti-tumor immune response is considered to be due to the tumor infiltrating lymphocytes that bind to tumor antigens, which can be either wild-type, early stem cell proteins, presumably foreign to a developed immune system; or mutant peptides, foreign to the immune system because of a mutant amino acid or an otherwise somatically altered amino acid sequence. Disclosed herein are novel methods for assessing the complementarity of tumor mutant peptides and complementarity determining regions (CDRs) of T cell receptors, B cell receptors, and antibodies, based on the retrieval of CDR3 amino acid sequences from both tumor specimen and patient blood exomes and by using a process of assessing CDR3s and mutant amino acid electrical charges. It is shown herein that high electrostatic complementarity and hydropathy values are associated with higher survival rates. In addition, the approach shown herein leads to the identification of genes contributing significantly to the complementary, TCR CDR3, mutant amino acids. The data shown herein indicate a novel approach to tumor immunoscoring and uses thereof for diagnosing, monitoring, and treating cancers. These methods are also used for the identification of high priority neo-antigen, peptide vaccines for treating cancers and/or to the identification of ex vivo stimulants of tumor infiltrating lymphocytes.

In some examples, disclosed herein is a method of determining overall survival probabilities in a subject with neuroblastoma, comprising:
a) obtaining a blood sample from the subject, wherein the blood sample comprises a T-cell receptor (TCR);
b) extracting complementarity determining region 3 (CDR3) amino acid (AA) sequences from the T-cell receptor (TCR) in the blood sample, thereby obtaining extracted CDR3 AA sequences;
c) identifying an exact match of extracted CDR3 AA sequences to known anti-viral CDR3 AA sequences from the blood sample; thereby obtaining an exact match anti-viral CDR3 AA sequence; and
d) correlating presence of the exact match anti-viral CDR3 AA sequence with overall survival probability of the subject,

wherein the presence of the exact match anti-viral CDR3 AA sequence in the blood sample is correlated to poor overall survival compared to a reference control, wherein the reference control does not have the exact match anti-viral CDR3 AA sequence in the blood sample.

In some examples, the known anti-viral CDR3 AA sequences of one or more viruses selected from the group consisting of Cytomegalovirus (CMV), Epstein-Barr virus (EBV), Hepatitis C virus (HCV), human immunodeficiency virus (HIV), Influenza A, and SARS-CoV-2.

In some examples, the T cell receptor (TCR) comprises an alpha chain or a beta chain.

In some examples, disclosed herein is a method of determining overall survival in a subject with neuroblastoma, comprising:
a) obtaining a sample from the subject, wherein the sample comprises a T-cell receptor (TCR);
b) extracting complementarity determining region 3 (CDR3) from the T-cell receptor (TCR) in the sample, thereby obtaining extracted CDR3;
c) obtaining a chemical complementarity score (CS) by interacting known viral antigens to the extracted CDR3 from the sample;
d) calculating the CS; and
e) correlating the CS with overall survival of the subject,

wherein a high CS is correlated to poor overall survival compared to a reference control, wherein the reference control has low CS.

In some examples, the high CS comprises a high chemical complementarity between the known viral antigens and the extracted CDR3.

In some examples, the CS is calculated using hydrophobic interactions, electrostatic interactions, or a combination thereof.

In some examples, the sample comprises blood or tumor biopsy.

In some examples, the known viral antigens of one or more viruses selected from the group consisting of Cytomegalovirus (CMV), Epstein-Barr virus (EBV), Hepatitis C virus (HCV), human immunodeficiency virus (HIV), Influenza A, and SARS-CoV-2.

In some examples, disclosed herein is a method of treating neuroblastoma in a subject, comprising:
a) obtaining a sample from the subject, wherein the sample comprises a T-cell receptor (TCR);
b) extracting complementarity determining region 3 (CDR3) amino acid (AA) sequences from the T-cell receptor (TCR) in the sample, thereby obtaining extracted CDR3 AA sequences;
c) identifying an exact match of extracted CDR3 AA sequences to anti-Cytomegalovirus (CMV) CDR3 AA sequences from the sample; thereby obtaining an exact match anti-CMV CDR3 AA sequence;
d) correlating presence of the exact match anti-CMV CDR3 AA sequence with overall survival of the subject; and
e) administering a therapeutically effective amount of an anti-CMV comprising Ganciclovir or Valganciclovir to the subject with a presence of the exact match anti-CMV CDR3 sequence,

wherein the presence of the exact match anti-CMV CDR3 AA sequence in the sample is correlated to poor overall survival.

In some examples, disclosed herein is a method of treating neuroblastoma in a subject, comprising:
a) obtaining a sample from the subject, wherein the sample comprises a T-cell receptor (TCR);
b) extracting complementarity determining region 3 (CDR3) from the T-cell receptor (TCR) in the sample, thereby obtaining extracted CDR3;
c) obtaining a chemical complementarity score (CS) by interacting CMV antigen to the extracted CDR3 from the sample;
d) calculating the CS;
e) correlating the CS with overall survival of the subject; and
f) administering a therapeutically effective amount of an anti-CMV comprising Ganciclovir or Valganciclovir to the subject with a high CS, wherein a high CS is correlated to poor overall survival.

In some examples, disclosed herein is a method of treating neuroblastoma in a subject based on correlation between chemical complementary scoring and gene amplification, comprising:
a) obtaining a sample from the subject, wherein the sample comprises a T-cell receptor (TCR);
b) extracting complementarity determining region 3 (CDR3) from the T-cell receptor (TCR) in the sample, thereby obtaining extracted CDR3;
c) obtaining a chemical complementarity score (CS) by interacting known viral antigen to the extracted CDR3 from the sample;
d) calculating the CS;
e) correlating the CS with overall survival of the subject; wherein a high CS is correlated to poor overall survival compared to a reference control, wherein the reference control has low CS;
f) determining gene amplification status of MYCN gene in the sample;
g) classifying the subject into a high-risk group based on the high CS and MYCN gene amplification in the sample; and
h) administering a therapeutically effective amount of an anti-viral comprising Ganciclovir or Valganciclovir to the high-risk group.

In some examples, disclosed herein is a method of treating neuroblastoma in a subject based on correlation between exact match sequencing and gene amplification, comprising:
a) obtaining a sample from the subject, wherein the sample comprises a T-cell receptor (TCR);
b) extracting complementarity determining region 3 (CDR3) amino acid (AA) sequences from the T-cell receptor (TCR) in the sample, thereby obtaining extracted CDR3 AA sequences;
c) identifying an exact match of extracted CDR3 AA sequences to known anti-viral CDR3 AA sequences from the sample; thereby obtaining an exact match anti-viral CDR3 AA sequence;
d) correlating presence of the exact match anti-viral CDR3 AA sequence with overall survival of the subject, wherein the presence of the exact match anti-viral CDR3 AA sequence in the sample is correlated to poor overall survival;
e) determining gene amplification status of MYCN gene in the sample;
f) classifying the subject into a high-risk group based on the presence of the exact match anti-viral CDR3 AA sequence and MYCN gene amplification in the sample; and
g) administering a therapeutically effective amount of an anti-viral comprising Ganciclovir or Valganciclovir to the high-risk group.

In some examples, disclosed herein is a kit for use in a method of detecting the presence of anti-viral T-cell receptors in a subject with cancer, comprising:
a) isolating a nucleic acid from a tumor sample derived from the subject;
b) sequencing a polynucleotide encoding a complementarity determining region (CDR) sequence of a T-cell receptor in the tumor microenvironment, thereby obtaining extracted CDR3 sequences;
c) identifying exact match anti-viral CDR3 sequences from the extracted CDR3 sequences, thereby obtaining the exact match anti-viral CDR3 sequences;
d) determining exact match anti-viral CDR3 sequences to known viral antigen sequences;
e) determining gene amplification status of MYCN gene in tumor cells of the subject; and
f) classifying the subject into a high-risk group using multivariate analysis based on the presence of both exact match anti-viral CDR3 sequences and MYCN gene amplification in the tumor cells.

In some examples, the CDR domain is a CDR3 domain.

In some examples, the cancer is selected from the group consisting of neuroblastoma, low-grade glioma, stomach adenocarcinoma, esophageal cancer, melanoma, lung squamous cell carcinoma, lung adenocarcinoma, breast cancer, cervical squamous cell carcinoma, bladder cancer, muscle invasive bladder cancer, and soft tissue sarcoma.

In some examples, the protein associated with the cancer is selected from the group consisting of MYC proto-oncogene neuroblastoma derived (MYCN), isocitrate dehydrogenase 1 (IDH1), Phosphatidylinositol-4,5-Bisphosphate 3-Kinase Catalytic Subunit Alpha (PIK3CA), B-Raf proto-oncogene (BRAF), Dynein heavy chain 9 (DNAH9), myosin heavy chain 1 (MYH1), Tenascin-R (TNR), Teneurin-1 (TNM1), Plexin-A4 (PLXNA4A), Microtubule-actin cross-linking factor 1 (MACF1), Tumor protein p53 (TP53), ATP-dependent helicase ATRX (ATRX), Neuroblastoma RAS viral oncogene homolog (NRAS), and Retinoblastoma protein (RB1).

Reference will now be made in detail to the examples of the invention, examples of which are illustrated in the drawings and the examples. This invention may, however, be embodied in many different forms and should not be construed as limited to the examples set forth herein.

Unless defined otherwise, all technical and scientific terms used herein have the same meaning as commonly understood to one of ordinary skill in the art to which this disclosure belongs.

### Terminology

Terms used throughout this application are to be construed with ordinary and typical meaning to those of ordinary skill in the art. However, Applicant desires that the following terms be given the particular definition as defined below.

As used herein, the article “a,” “an,” and “the” means “at least one,” unless the context in which the article is used clearly indicates otherwise.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

The terms “about” and “approximately” are defined as being “close to” as understood by one of ordinary skill in the art. In one non-limiting embodiment, the terms are defined to be within 10%. In another non-limiting embodiment, the terms are defined to be within 5%. In still another non-limiting embodiment, the terms are defined to be within 1%.

According to the present invention, “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (1) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). “Complementarity Determining Regions” or “CDRs” refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, includes six CDRs, comprising the CDR set from each of a heavy and a light chain V region. CDR3s are most variable, of which the tertiary structure determines antigen recognition of an antibody. Framework Regions (FRs) refer to amino acid sequences interposed between CDRs.

As used herein, the term “antibody or a functional fragment thereof” encompasses chimeric antibodies and hybrid antibodies, with dual or multiple antigen or epitope specificities, and fragments, such as F(ab′)2, Fab′, Fab, Fv, scFv, and the like, including hybrid fragments. Thus, fragments of the antibodies that retain the ability to bind their specific antigens are provided. For example, fragments of antibodies which maintain antigen recognition property are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or functional fragments thereof” are conjugates of antibody fragments and antigen binding proteins (single chain antibodies). The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

The term “cancer” or “neoplasms” used herein meant to include all types of cancerous growths or oncogenic processes, metastatic tissues or malignantly transformed cells, tissues, or organs, irrespective of histopathologic type or stage of invasiveness. The terms “cancer” or “neoplasms” include malignancies of the various organ systems, such as malignancies affecting skin, brain, spinal cord, cervix, bladder, lung, breast, thyroid, lymphoid tissues, connecting tissues, gastrointestinal, and genito-urinary tracts, that include, but are not limited to, glioma, melanoma, lung cancer, breast cancer, cervical squamous cell carcinoma, bladder cancer, and soft tissue sarcoma. The term “cancer metastasis” has its general meaning in the art and refers to the spread of a tumor from one organ or part to another non-adjacent organ or part.

The term “comprising” and variations thereof as used herein is used synonymously with the term “including” and variations thereof and are open, non-limiting terms. Although the terms “comprising” and “including” have been used herein to describe various examples, the terms “consisting essentially of” and “consisting of” can be used in place of “comprising” and “including” to provide for more specific examples and are also disclosed.

“Complementarity Determining Regions” or “CDRs” refer to amino acid sequences which together define the binding affinity and specificity of the natural variable domain of a native binding site of an antibody, a BCR, or a TCR. The extent of CDRs have been precisely defined and identified by methods known in the arts, such as “Sequences of Proteins of Immunological Interest,” E. Kabat et al., U.S. Department of Health and Human Services, (1991); Wu T T, Kabat E A. “An analysis of the sequences of the variable regions of Bence Jones proteins and myeloma light chains and their implications for antibody complementarity”. J Exp Med (1970); “Canonical structures for the hypervariable regions of immunoglobulins”. Chothia C, Lesk A M J Mol Biol. 1987 Aug. 20; 196(4):901-17, which are incorporated herein by reference for all purposes. In some examples, a CDR begins by the second cysteine in the variable domain, and at the end by the first amino acid in the conserved Phe/Trp-Gly-X-Gly J-region motif.

A “composition” is intended to include a combination of active agent and another compound or composition, inert (for example, a detectable agent or label) or active, such as an adjuvant.

As used herein, the terms “determining,” “measuring,” and “assessing,” and “assaying” are used interchangeably and include both quantitative and qualitative determinations.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein the term “encoding” refers to the inherent property of specific sequences of nucleotides in a nucleic acid, to serve as templates for synthesis of other molecules having a defined sequence of nucleotides (i.e. rRNA, tRNA, other RNA molecules) or amino acids and the biological properties resulting therefrom.

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the functional fragment must possess a bioactive property, such as antigen binding and antigen recognition.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The term “isolating” as used herein refers to isolation from a biological sample, i.e., blood, plasma, tissues, exosomes, or cells. As used herein the term “isolated,” when used in the context of, e.g., a nucleic acid, refers to a nucleic acid of interest that is at least 60% free, at least 75% free, at least 90% free, at least 95% free, at least 98% free, and even at least 99% free from other components with which the nucleic acid is associated with prior to purification.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22: 1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length Win the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

The term “specific binding” refers to the ability of an antigen-binding protein (e.g., an antibody) to preferentially bind to a particular analyte that is present in a homogeneous mixture of different analytes. In certain examples, a specific binding interaction will discriminate between desirable and undesirable antigen in a sample, in some examples more than about 10 to 100-fold or more (e.g., more than about 1000- or 10,000-fold). In certain examples, the affinity between an antigen-binding protein (e.g., an antibody, TCR, or BCR) and an antigen when they are specifically bound in an antigen-binding protein/antigen complex is characterized by a KD (dissociation constant) of less than 10-6 M, less than 10-7 M, less than 10-8 M, less than 10-9 M, less than 10-9 M, less than 10-11 M, or less than about 10-12 M.

The term “subject” or “host” refers to any individual who is the target of administration or treatment. The subject can be a vertebrate, for example, a mammal. Thus, the subject can be a human or veterinary patient. The term “patient” refers to a subject under the treatment of a clinician, e.g., physician. The subject can be either male or female.

“T cell receptor” or “TCR” refers to an immunoglobulin superfamily member (having a variable binding domain, a constant domain, a transmembrane region, and a short cytoplasmic tail; see, e.g., Janeway et al., Immunobiology: The Immune System in Health and Disease, 3rd Ed., Current Biology Publications, p. 4:33, 1997) capable of specifically binding to an antigen peptide bound to a major histocompatibility complex (MHC). A TCR can be found on the surface of a cell and generally is comprised of a heterodimer having a and β chains (also known as TCRα and TCRβ, respectively), or γ and δ chains (also known as TCRγ and TCRδ of a γδTCR, respectively). Like immunoglobulins, the extracellular portion of TCR chains (e.g., α-chain, β-chain) contain two immunoglobulin domains, a variable domain (e.g., α-chain variable domain or Va, β-chain variable domain or Vb) at the N-terminus, and one constant domain (e.g., a-chain constant domain or Ca, β-chain constant domain or Cb) adjacent to the cell membrane. Also, like immunoglobulins, the variable domains contain complementary determining regions (CDRs) separated by framework regions (FRs). For an αβTCR, the alpha chain and beta chain each have three CDRs, wherein CDR3 is the most variable of which the tertiary structure determines antigen recognition. The source of a TCR as used in the present disclosure may be from various animal species, such as a human, mouse, rat, rabbit or other mammal.

The term “tissue” refers to a group or layer of similarly specialized cells which together perform certain special functions. The term “tissue” is intended to include, blood, blood preparations such as plasma and serum, bones, joints, muscles, smooth muscles, lung tissues, and organs.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder (e.g., a cancer), or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition (e.g. cancer). Therapeutically effective and prophylactically effective amounts of a given therapeutic agent will typically vary with respect to factors such as the type and severity of the disorder or disease being treated and the age, gender, and weight of the subject.

The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Methods of Diagnosing and Treating Cancers

In some examples, disclosed herein is a method of determining overall survival probabilities in a subject with neuroblastoma, comprising:
a) obtaining a blood sample from the subject, wherein the blood sample comprises a T-cell receptor (TCR);
b) extracting complementarity determining region 3 (CDR3) amino acid (AA) sequences from the T-cell receptor (TCR) in the blood sample, thereby obtaining extracted CDR3 AA sequences;
c) identifying an exact match of extracted CDR3 AA sequences to known anti-viral CDR3 AA sequences from the blood sample; thereby obtaining an exact match anti-viral CDR3 AA sequence; and
d) correlating presence of the exact match anti-viral CDR3 AA sequence with overall survival probability of the subject,

wherein the presence of the exact match anti-viral CDR3 AA sequence in the blood sample is correlated to poor overall survival compared to a reference control, wherein the reference control does not have the exact match anti-viral CDR3 AA sequence in the blood sample.

In some examples, the known anti-viral CDR3 AA sequences of one or more viruses selected from the group consisting of Cytomegalovirus (CMV), Epstein-Barr virus (EBV), Hepatitis C virus (HCV), human immunodeficiency virus (HIV), Influenza A, and SARS-CoV-2.

In some examples, the T cell receptor (TCR) comprises an alpha chain or a beta chain.

In some examples, disclosed herein is a method of determining overall survival in a subject with neuroblastoma, comprising:
a) obtaining a sample from the subject, wherein the sample comprises a T-cell receptor (TCR);
b) extracting complementarity determining region 3 (CDR3) from the T-cell receptor (TCR) in the sample, thereby obtaining extracted CDR3;
c) obtaining a chemical complementarity score (CS) by interacting known viral antigens to the extracted CDR3 from the sample;
d) calculating the CS; and
e) correlating the CS with overall survival of the subject,

wherein a high CS is correlated to poor overall survival compared to a reference control, wherein the reference control has low CS.

In some examples, the high CS comprises a high chemical complementarity between the known viral antigens and the extracted CDR3.

In some examples, the CS is calculated using hydrophobic interactions, electrostatic interactions, or a combination thereof.

In some examples, the sample comprises blood or tumor biopsy.

“CMV” is a β-herpesvirus. Human cytomegalovirus (HCMV) is unique in the human population, and it has been reported that it usually does not cause clinical disease except in immunocompromised hosts. Several human herpesviruses are involved in a number of human malignancies including lymphoma, nasopharyngeal cancer, cervical cancer and Kaposi's sarcoma. Recently, it has been reported that the expression and detection of intact viral HCMV antigens occurs in several tumors.

In some examples, disclosed herein is a method of treating neuroblastoma in a subject, comprising:
a) obtaining a sample from the subject, wherein the sample comprises a T-cell receptor (TCR);
b) extracting complementarity determining region 3 (CDR3) amino acid (AA) sequences from the T-cell receptor (TCR) in the sample, thereby obtaining extracted CDR3 AA sequences;
c) identifying an exact match of extracted CDR3 AA sequences to anti-Cytomegalovirus (CMV) CDR3 AA sequences from the sample; thereby obtaining an exact match anti-CMV CDR3 AA sequence;
d) correlating presence of the exact match anti-CMV CDR3 AA sequence with overall survival of the subject; and
e) administering a therapeutically effective amount of an anti-CMV comprising Ganciclovir or Valganciclovir to the subject with a presence of the exact match anti-CMV CDR3 sequence,

wherein the presence of the exact match anti-CMV CDR3 AA sequence in the sample is correlated to poor overall survival.

In some examples, disclosed herein is a method of treating neuroblastoma in a subject, comprising:
a) obtaining a sample from the subject, wherein the sample comprises a T-cell receptor (TCR);
b) extracting complementarity determining region 3 (CDR3) from the T-cell receptor (TCR) in the sample, thereby obtaining extracted CDR3;
c) obtaining a chemical complementarity score (CS) by interacting CMV antigen to the extracted CDR3 from the sample;
d) calculating the CS;
e) correlating the CS with overall survival of the subject; and
f) administering a therapeutically effective amount of an anti-CMV comprising Ganciclovir or Valganciclovir to the subject with a high CS, wherein a high CS is correlated to poor overall survival.

Examples of anti-viral agents include, but are not limited to, ganciclovir (e.g., CYTOVENE™), valganciclovir (e.g. VALCYTE™), foscarnet (e.g. FOSCAVIR™) Cidofovir (e.g. VISTIDE™), HPMPC), adefovir (e.g., PMEA, PREVEON™, HEPSERA™) acyclovir (e.g., ZOVIRAX™), valacyclovir (e.g. VALTREX™, ZELITREX™), polyanions, and protein kinase C inhibitors (eg, bis-indolemaleide).

In one embodiment, the anti-viral agent used in combination with the compositions and methods of the present invention is ganciclovir, valganciclovir, cidofovir or foscarnet.

In some examples, the chemical complementarity scores are calculated using hydrophobic interactions, electrostatic interactions, or a combination thereof.

In some examples, disclosed herein is a method further comprising determination of worse overall survival in neuroblastoma subjects with tumor MYCN gene amplification and the presence of CMV-TCR CDR3 sequences.

In some examples, disclosed herein is a method of treating neuroblastoma in a subject based on correlation between chemical complementary scoring and gene amplification, comprising:
a) obtaining a sample from the subject, wherein the sample comprises a T-cell receptor (TCR);
b) extracting complementarity determining region 3 (CDR3) from the T-cell receptor (TCR) in the sample, thereby obtaining extracted CDR3;
c) obtaining a chemical complementarity score (CS) by interacting known viral antigen to the extracted CDR3 from the sample;
d) calculating the CS;
e) correlating the CS with overall survival of the subject; wherein a high CS is correlated to poor overall survival compared to a reference control, wherein the reference control has low CS;
f) determining gene amplification status of MYCN gene in the sample;
g) classifying the subject into a high-risk group based on the high CS and MYCN gene amplification in the sample; and
h) administering a therapeutically effective amount of an anti-viral comprising Ganciclovir or Valganciclovir to the high-risk group.

In some examples, disclosed herein is a method of treating neuroblastoma in a subject based on correlation between exact match sequencing and gene amplification, comprising:
a) obtaining a sample from the subject, wherein the sample comprises a T-cell receptor (TCR);
b) extracting complementarity determining region 3 (CDR3) amino acid (AA) sequences from the T-cell receptor (TCR) in the sample, thereby obtaining extracted CDR3 AA sequences;
c) identifying an exact match of extracted CDR3 AA sequences to known anti-viral CDR3 AA sequences from the sample; thereby obtaining an exact match anti-viral CDR3 AA sequence;
d) correlating presence of the exact match anti-viral CDR3 AA sequence with overall survival of the subject, wherein the presence of the exact match anti-viral CDR3 AA sequence in the sample is correlated to poor overall survival;
e) determining gene amplification status of MYCN gene in the sample;
f) classifying the subject into a high-risk group based on the presence of the exact match anti-viral CDR3 AA sequence and MYCN gene amplification in the sample; and
g) administering a therapeutically effective amount of an anti-viral comprising Ganciclovir or Valganciclovir to the high-risk group.

In some examples, the CDR domain is a CDR3 domain.

In some examples, the cancer is selected from the group consisting of neuroblastoma, low-grade glioma, stomach adenocarcinoma, esophageal cancer, melanoma, lung squamous cell carcinoma, lung adenocarcinoma, breast cancer, cervical squamous cell carcinoma, bladder cancer, muscle invasive bladder cancer, and soft tissue sarcoma.

In some examples, the protein associated with the cancer is selected from the group consisting of MYC proto-oncogene, neuroblastoma derived (MYCN), isocitrate dehydrogenase 1 (IDH1), Phosphatidylinositol-4,5-Bisphosphate 3-Kinase Catalytic Subunit Alpha (PIK3CA), B-Raf proto-oncogene (BRAF), Dynein heavy chain 9 (DNAH9), myosin heavy chain 1 (MYH1), Tenascin-R (TNR), Teneurin-1 (TNM1), Plexin-A4 (PLXNA4A), Microtubule-actin cross-linking factor 1 (MACF1), Tumor protein p53 (TP53), ATP-dependent helicase ATRX (ATRX), Neuroblastoma RAS viral oncogene homolog (NRAS), and Retinoblastoma protein (RB1).

As noted above, “Complementarity Determining Regions” or “CDRs” refer to amino acid sequences which together define the binding affinity and specificity of the natural Fv region of a native binding site of an immunoglobulin or a TCR. The light (L) and heavy (H) chains of an immunoglobulin each have three CDRs, designated L-CDR1, L-CDR2, L-CDR3 and H-CDR1, H-CDR2, H-CDR3, respectively. For an αβTCR, the alpha chain and beta chain each have three CDRs. Accordingly, in some examples, the CDR is a CDR1 of a light chain of an antibody. In some examples, the CDR is a CDR2 of a light chain of an antibody. In some examples, the CDR is a CDR3 of a light chain of an antibody. In some examples, the CDR is a CDR1 of a heavy chain of an antibody. In some examples, the CDR is a CDR2 of a heavy chain of an antibody.

In some examples, the CDR is a CDR1 of an alpha chain of a TCR. In some examples, the CDR is a CDR2 of an alpha chain of a TCR. In some examples, the CDR is a CDR3 of an alpha chain of a TCR. In some examples, the CDR is a CDR1 of a beta chain of a TCR. In some examples, the CDR is a CDR2 of a beta chain of a TCR. In some examples, the CDR is a CDR3 of a beta chain of a TCR.

In some examples, disclosed herein is a method for predicting the CDR is a CDR of a γ chain of a γδTCR. In some examples, the CDR is a CDR of a δ chain of a γδTCR.

In some examples, the nucleic acid is any preceding aspect is a DNA or an RNA. In some examples, the nucleic acid is a DNA. In some examples, the nucleic acid is an RNA. In some examples, the polynucleotide is a DNA. In some examples, the polynucleotide is an RNA. In some examples, the DNA comprises an exon and an intron. In some examples, the DNA is an exon.

It should be understood and herein contemplated that a polypeptide's net charge depends on the number of the charged amino acids the polypeptide contains and the pH of the environment. At physiological pH (pH 7.4), for example, five amino acid residues out of the 20 common amino acids can be charged: two are negative charged: aspartic acid (Asp, D) and glutamic acid (Glu, E) (acidic side chains), and three are positive charged: lysine (Lys, K), arginine (Arg, R) and histidine (His, H) (basic side chains). The term “net charge per residue” of a polypeptide (e.g. a CDR domain) in a certain pH environment is calculated as dividing the overall charge of the polypeptide in such pH environment by the number of amino acid residues of the polypeptide.

In some examples, the reference control is the complementarity score of a CDR domain and a protein associated with a cancer for the lowest 10%, 20%, 30%, 40%, or 50% of the complementarity score of a reference population of patient samples having the cancer. In some examples, the reference control is the complementarity score of a CDR domain and a protein associated with a cancer for the highest 10%, 20%, 30%, 40%, or 50% of the complementarity score of a reference population of patient samples having the cancer. Accordingly, the subject has a shorter overall survival if the complementarity score is lower in the biological sample derived from the subject compared to the reference control, and the subject has a longer overall survival if the complementarity score is higher in the biological sample derived from the subject compared to a reference control.

In some examples, the subject as having a shorter overall survival comprises the subject having an overall survival of about 1 month or less, about 2 months or less, about 4 months or less, about 6 months or less, about 8 months or less, about 10 months or less, about 12 months or less, about 14 months or less, about 16 months or less, about 18 months or less, about 20 months or less, about 22 months or less, about 25 months or less, or about 30 months or less, about 35 months or less, about 40 months or less, about 45 months or less, about 50 months or less, about 55 months or less, about 60 months or less, about 65 months or less, about 70 months or less, about 75 months or less, about 80 months or less, about 85 months or less, about 90 months or less, about 95 months or less, about 100 months or less, about 150 months or less, about 200 months or less, or about 250 months or less.

In some examples, the subject is a human. In some examples, the human has or is suspected of having a cancer. In some examples, the cancer is selected from the group consisting of neuroblastoma, low-grade glioma, stomach adenocarcinoma, esophageal cancer, melanoma, lung squamous cell carcinoma, lung adenocarcinoma, breast cancer, cervical squamous cell carcinoma, bladder cancer, muscle invasive bladder cancer, and soft tissue sarcoma.

The term “protein associated with a cancer” of any preceding aspect refers to a protein that has the potential to cause cancer. In tumor cells, the gene encoding such a protein may be mutated, overly expressed, or underly expressed. This term can also refer to a protein that is mutated, overly expressed, or underly expressed in a cancer cell or a cell undergoing oncogenic processes. In some examples, the protein associated with a cancer is selected from the group consisting of isocitrate dehydrogenase 1 (IDH1) (External Ids: HGNC: 5382; Entrez Gene: 3417; Ensembl: ENSG00000138413; OMIM: 147700; UniProtKB: 075874), Phosphatidylinositol-4,5-Bisphosphate 3-Kinase Catalytic Subunit Alpha (PIK3CA, External Ids: HGNC: 8975; Entrez Gene: 5290; Ensembl: ENSG00000121879; OMIM: 171834; UniProtKB: P42336), Inositol 1,4,5-trisphosphate receptor, type 2 (ITPR2, External Ids: HGNC: 6181; Entrez Gene: 3709; Ensembl: ENSG00000123104; OMIM: 600144; UniProtKB: Q14571), B-Raf proto-oncogene (BRAF, External Ids: HGNC: 1097; Entrez Gene: 673; Ensembl: ENSG00000157764; OMIM: 164757; UniProtKB: P15056), Dynein heavy chain 9 (DNAH9, External Ids: HGNC: 2953; Entrez Gene: 1770; Ensembl: ENSG00000007174; OMIM: 603330; UniProtKB: Q9NYC9), myosin heavy chain 1 (MYH1, External Ids: HGNC: 7567; Entrez Gene: 4619; Ensembl: ENSG00000109061; OMIM: 160730; UniProtKB: P12882), Tenascin-R (TNR, External Ids: HGNC: 11953; Entrez Gene: 7143; Ensembl: ENSG00000116147; OMIM: 601995; UniProtKB: Q92752), Teneurin-1 (TNM1; HGNC: 8117; Entrez Gene: 10178; Ensembl: ENSG00000009694; OMIM: 300588; UniProtKB: Q9UKZ4), Plexin-A4 (PLXNA4A, External Ids: HGNC: 9102; Entrez Gene: 91584; Ensembl: ENSG00000221866; OMIM: 604280; UniProtKB: Q9HCM2), Microtubule-actin cross-linking factor 1 (MACF1, External Ids: HGNC: 13664; Entrez Gene: 23499; Ensembl: ENSG00000127603; OMIM: 608271; UniProtKB: Q9UPN3), Tumor protein p53 (TP53, External Ids: HGNC: 11998; Entrez Gene: 7157; Ensembl: ENSG00000141510; OMIM: 191170; UniProtKB: P04637), ATP-dependent helicase ATRX (ATRX, External Ids; HGNC: 886; Entrez Gene: 546; Ensembl: ENSG00000085224; OMIM: 300032; UniProtKB: P46100), Neuroblastoma RAS viral oncogene homolog (NRAS, External Ids: HGNC: 7989; Entrez Gene: 4893; Ensembl: ENSG00000213281; OMIM: 164790; UniProtKB: P01111) and Retinoblastoma protein (RB1, External Ids: HGNC: 9884; Entrez Gene: 5925; Ensembl: ENSG00000139687; OMIM: 614041; UniProtKB: P06400), or a functional fragment thereof.

Accordingly, in some examples, a complementarity score that is less than 0 denotes the subject as having a longer overall survival. Accordingly, in some examples, the subject is administered with a therapeutically effective amount of an anti-cancer agent if the complementarity score is more than 0.

In some examples, wherein the protein associated with a cancer is selected from the group consisting of MYC proto-oncogene, neuroblastoma derived (MYCN), isocitrate dehydrogenase 1 (IDH1), Phosphatidylinositol-4,5-Bisphosphate 3-Kinase Catalytic Subunit Alpha (PIK3CA), B-Raf proto-oncogene (BRAF), Dynein heavy chain 9 (DNAH9), myosin heavy chain 1 (MYH1), Tenascin-R (TNR), Teneurin-1 (TNM1), Plexin-A4 (PLXNA4A), Microtubule-actin cross-linking factor 1 (MACF1), Tumor protein p53 (TP53), ATP-dependent helicase ATRX (ATRX), Neuroblastoma RAS viral oncogene homolog (NRAS), and Retinoblastoma protein (RB1).

In some examples, the anti-cancer agent is selected from the group consisting of cordycepin, fenretinide, Zyclara, vemurafenib (Zelboraf®), dabrafenib (Tafinlar™) encorafenib (Braftovi™), pembrolizumab (Keytruda), nivolumab (Opdivo), Anthracyclines, Taxanes, 5-fluorouracil (5-FU), Cyclophosphamide (Cytoxan), Carboplatin (Paraplatin), cisplatin, carboplatin, Vinorelbine (Navelbine), Capecitabine (Xeloda), Gemcitabine (Gemzar), Ixabepilone (Ixempra), Eribulin (Halaven), Fulvestrant (Faslodex), Letrozole (Femara), Anastrozole (Arimidex), exemestane (Aromasin), Trastuzumab (Herceptin), Pertuzumab (Perjeta), Ado-trastuzumab emtansine, Lapatinib (Tykerb), Neratinib (Nerlynx), Everolimus (Afinitor), Olaparib (Lynparza), talazoparib (Talzenna), Alpelisib (Piqray), Atezolizumab (Tecentriq), Paclitaxel (Taxol), Albumin-bound paclitaxel (nab-paclitaxel, Abraxane), Docetaxel (Taxotere), Etoposide (VP-16), Pemetrexed (Alimta), Bevacizumab (Avastin), Ramucirumab (Cyramza), ifosfamide (Ifex™), irinotecan (Camptosar™) mitomycin, doxorubicin (Adriamycin), methotrexate, vinblastine (CMV), durvalumab (Imfinzi™), avelumab (Bavencio™), Erdafitinib (Balversa), dacarbazine (DTIC), epirubicin, temozolomide (Temodar™), gemcitabine (Gemzar™), trabectedin (Yondelis™), and Pazopanib (Votrient).

As would be apparent, the sequencing may be done using a next generation sequencing platform, e.g., Illumina's reversible terminator method, Roche's pyrosequencing method, Life Technologies' sequencing by ligation (the SOLiD platform) or Life Technologies' Ion Torrent platform, etc. Examples of such methods are described in the following references: Margulies et al (Nature 2005 437: 376-80); Ronaghi et al (Analytical Biochemistry 1996 242: 84-9); Shendure (Science 2005 309: 1728); Imelfort et al (Brief Bioinform. 2009 10:609-18); Fox et al (Methods Mol Biol. 2009; 553:79-108); Appleby et al (Methods Mol Biol. 2009; 513:19-39) and Morozova (Genomics. 2008 92:255-64), which are incorporated by reference for the general descriptions of the methods and the particular steps of the methods, including all starting products, reagents, and final products for each of the steps. In other examples, the sequencing may be done using nanopore sequencing (e.g. as described in Soni et al Clin Chem 53: 1996-2001 2007, or as described by Oxford Nanopore Technologies).

### EXAMPLES

The following examples are set forth below to illustrate the compounds, systems, methods, and results according to the disclosed subject matter. These examples are not intended to be inclusive of all examples of the subject matter disclosed herein, but rather to illustrate representative methods and results. These examples are not intended to exclude equivalents and variations of the present invention which are apparent to one skilled in the art.

### Example 1. Determine the Presence of Anti-Viral CDR3s in the Blood is Associated with OS Probability Distinctions

Anti-viral CDR3s represent specific viruses. One hundred thousand six-hundred forty-six recombination reads for TRA and TRB were extracted from the TARGET-NBL WXS files, representing 205 blood samples (Table 1). The CDR3s from the TRA and TRB recombination reads from the blood WXS files were then searched for an exact amino acid (AA) sequence match to known anti-viral CDR3s (Methods). To determine whether the presence of anti-viral CDR3s in the blood was associated with OS probability distinctions, patient IDs having anti-viral CDR3s to those without, for the following viruses: CMV, EBV, HCV, HIV, Influenza A, and SARS-CoV-2 were compared. Results indicated that, for every virus, the presence of anti-viral CDR3s represented a worse OS outcome probability (Table 1).

### Table 1. OS Probability Distinctions Based on the Presence of Anti-Viral CDR3s for Various Viruses

| | Median survival | Median survival | |
|---|---|---|---|
| | for cases with | for cases with | |
| | at least one | no anti-viral | |
| Virus | anti-viral CDR3 | CDR3s recovered | |
| representing | recovered from | from the blood | |
| the anti-viral | the blood WXS | WXS file | Log-rank |
| CDR3s | file (months) | (months) | p value |
| CMV | 29 | 73 | 7.759e−6 |
| EBV | 22 | 51 | 1.523e−4 |
| HCV | 27 | 46 | 0.0249 |
| HIV | 11 | 46 | 2.434e−3 |
| Influenza A | 31 | 51 | 1.029e−3 |
| SARS-CoV-2 | 27 | 54 | 2.286e−4 |

### Example 2. MYCN Amplification and the Detection of the Blood-Based, Anti-CMV TCR CDR3s

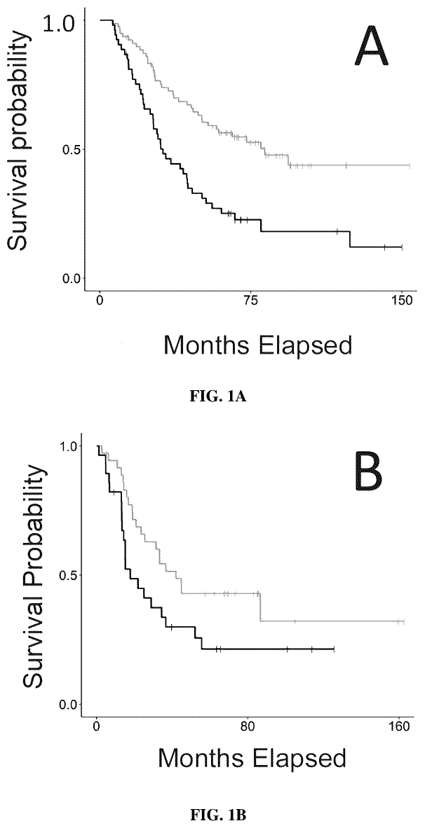

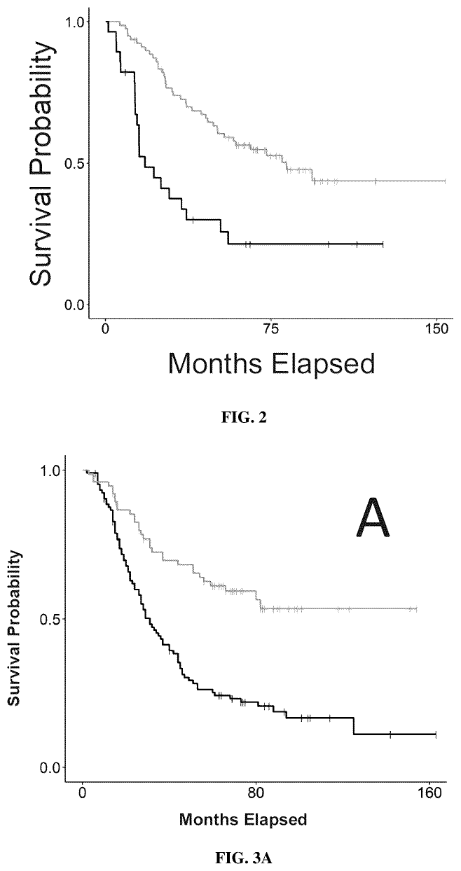

MYCN amplification is known to be associated with a poor NBL OS probability. Thus, case IDs were compared based on both amplification status and anti-viral CDR3 detection. For those cases that were positive for the anti-CMV CDR3s, there was no significant difference in OS based on MYCN amplification. In contrast, cases that did not represent MYCN amplification and were positive for anti-CMV CDR3s indicated a worse OS probability when compared to those without MYCN amplification and negative for anti-CMV CDR3s (FIG. 1A). Additionally, among cases with MYCN amplification, those that were positive for anti-CMV CDR3s had a worse OS probability than those cases that did not have anti-CMV CDR3s (FIG. 1B). Finally, there was an indication of the combined status of anti-viral CDR3 detection and MYCN amplification representing a very high risk of a poor OS probability (FIG. 2), i.e., in comparison to cases with neither anti-CMV or MYCN amplification. A summary of the FIGS. 1A and 1B, and FIG. 2 log rank p-values is in Table 2.

### Table 2. OS Probability Distinctions Based on Ani-CMV CDR3s Specific to the MYCN Amplification Status.

| | Median survival | Median survival | |
|---|---|---|---|
| | for cases with | for cases with | |
| | at least one | no anti- viral | |
| | anti-viral CDR3 | CDR3s recovered | |
| | recovered from | from the blood | |
| | the blood WXS | WXS file | |
| | file specific to | specific to | Log-rank |
| | CMV (months) | CMV (months) | p-value |
| Amplified | 18 | 43 | 0.0576 |
| Not amplified | 32 | 82 | 1.074e−4 |

### Example 3. Anti-CMV CDR3s from NBL Samples Tumors and Chemical Complementarity Scoring of the CDR3-CMV Antigen Pairs

The results above and the consistent evidence of CMV presence in NBL tumors led to the exploration of the possibility to detect anti-CMV CDR3s in tumor samples as represented by tumor WXS files. To determine whether the mining of such anti-CMV CDR3s form the NBL tumor WXS files could be used to distinguish survival probabilities, as was the case for the analogous analysis of the blood WXS files. However, an exact match of anti-CMV CDR3 AA sequences, to the TCR CDR3 AA sequences recovered from the NBL tumor WXS files did not yield such a distinction, possible owing to the much lower numbers of anti-CMV CDR3 recoveries from the tumor WXS files, i.e., lower numbers of anti-CMV CDR3 recoveries representing exact AA sequence matches to the known anti-CMV CDR3 AA sequences. Thus, the NBL blood and tumor CDR3s for chemical CSs was assessed, representing CDR3-CMV antigen combinations. The NBL cases were assessed for survival probability distinctions based on dividing the cases into groups of upper and lower 50th percentiles for the CSs, with high CSs representing higher chemical complementarity of the TCR CDR3-CMV antigens. These analyses were facilitated by the use of the adaptivematch.com web tool. Four CMV antigens, defined as such by the immune epitope database were tested, with three different chemical complementarity algorithms, with the three algorithms based on electrostatic interactions, hydrophobic interactions, and a combination of both hydrophobic and electrostatic interactions, including input files available for use at adaptivematch.com). For all CS approaches, and for all CMV antigens, the cases representing the upper 50th percentile of CDR3-CMV antigen CSs, for the blood-based CDR3s, represented highly significant reductions in survival probabilities (Tables 3-7). However, in the case of the tumor-based CDR3s, results were mixed, with only a subset CS approaches and certain antigens representing survival probabilities distinctions.

Table 3. OS Probabilities Associated with Chemical Complementary of Blood-Based CDR3s and the CMV Antigen, UL40.

CS, complementarity score (Methods); KM, Kaplan-Meier; WXS, whole exome sequence; TCR, T-cell receptor; CDR3, complementarity determining region-3.

Note: In the case of the Electrostatic CS groupings, the upper 50^{th} percentile group is established by the CS value until the CS changes to a lower value and at least 50% of the cases have been assigned to the upper group. Thus, in all cases, there are more cases representing the upper 50^{th} percentile of the Electrostatic CS values. The two other CS approaches represent more precise CSs and thus rarely have different numbers of cases in the two CS groups.

| | Electrostatic CS | | Combo CS | | Hydrophobicity CS | |
|---|---|---|---|---|---|---|
| | | Primary | | Primary | | Primary |
| | Blood WXS | Tumor WXS | Blood WXS | Tumor WXS | Blood WXS | Tumor WXS |
| | files as the | files as the | files as the | files as the | files as the | files as the |
| | source of the | source of the | source of the | source of the | source of the | source of the |
| | TCR CDR3s | TCR CDR3s | TCR CDR3s | TCR CDR3s | TCR CDR3s | TCR CDR3s |
| Cox-univariate | 3.16E−06 | 0.0037 | 0.0001 | 0.0200 | 0.0001 | 0.0624 |
| regression | | | | | | |
| p-value | | | | | | |
| KM, log-rank | 0.0026 | 0.0001 | 2.79E−05 | 0.5592 | 0.009280 | 0.1844 |
| p-value | | | | | | |
| KM median | 995 | 875 | 915 | 1049 | 954 | 995 |
| months upper | | | | | | |
| 50^{th} percentile | | | | | | |
| CS group | | | | | | |
| KM median | 1765 | 2384 | 2491 | 1314 | 1765 | 1330 |
| months upper | | | | | | |
| 50th percentile | | | | | | |
| CS group | | | | | | |
| KM median | −770 | −1509 | −1576 | −265 | −811 | −335 |
| survival | | | | | | |
| difference | | | | | | |
| (months) | | | | | | |
| N upper 50^{th} | 104 | 77 | 102 | 75 | 102 | 75 |
| percentile | | | | | | |
| CS group | | | | | | |
| N lower 50^{th} | 100 | 72 | 102 | 74 | 102 | 74 |
| percentile | | | | | | |
| CS group | | | | | | |

Table 4. OS Probabilities Associated with Chemical Complementary of CDR3s and the CMV Antigen, Pp65.

CS, complementarity score (Methods); KM, Kaplan-Meier; WXS, whole exome sequence; TCR, T-cell receptor; CDR3, complementarity determining region-3.

| | Electrostatic CS | | Combo CS | | Hydrophobicity CS | |
|---|---|---|---|---|---|---|
| | | Primary | | Primary | | Primary |
| | Blood WXS | Tumor WXS | Blood WXS | Tumor WXS | Blood WXS | Tumor WXS |
| | files as the | files as the | files as the | files as the | files as the | files as the |
| | source of the | source of the | source of the | source of the | source of the | source of the |
| | TCR CDR3s | TCR CDR3s | TCR CDR3s | TCR CDR3s | TCR CDR3s | TCR CDR3s |
| Cox-univariate | 4.78E−06 | 0.0006570 | 1.88E−05 | 0.01265 | 0.0001357 | 0.04146 |
| regression | | | | | | |
| p-value | | | | | | |
| KM, log-rank | 0.0008995 | 0.006264 | 0.0002295 | 0.1638 | 0.006862 | 0.3373 |
| p-value | | | | | | |
| KM median | 919 | 915 | 878 | 954 | 934 | 1049 |
| months upper | | | | | | |
| 50^{th} percentile | | | | | | |
| CS group | | | | | | |
| KM median | 2002 | 1543 | 2039 | 1314 | 1795 | 1193 |
| months upper | | | | | | |
| 50th percentile | | | | | | |
| CS group | | | | | | |
| KM median | −1083 | −628 | −1161 | −360 | −861 | −144 |
| survival | | | | | | |
| difference | | | | | | |
| (months) | | | | | | |
| N upper 50^{th} | 110 | 76 | 102 | 75 | 102 | 75 |
| percentile | | | | | | |
| CS group | | | | | | |
| N lower 50^{th} | 94 | 73 | 102 | 74 | 102 | 74 |
| percentile | | | | | | |
| CS group | | | | | | |

Table 5. OS Probabilities Associated with Chemical Complementary of CDR3s and the CMV Antigen, IE1.

CS, complementarity score (Methods); KM, Kaplan-Meier; WXS, whole exome sequence; TCR, T-cell receptor; CDR3, complementarity determining region-3.

| | Electrostatic CS | | Combo CS | | Hydrophobicity CS | |
|---|---|---|---|---|---|---|
| | | Primary | | Primary | | Primary |
| | Blood WXS | Tumor WXS | Blood WXS | Tumor WXS | Blood WXS | Tumor WXS |
| | files as the | files as the | files as the | files as the | files as the | files as the |
| | source of the | source of the | source of the | source of the | source of the | source of the |
| | TCR CDR3s | TCR CDR3s | TCR CDR3s | TCR CDR3s | TCR CDR3s | TCR CDR3s |
| Cox-univariate | 8.07E−06 | 0.004582 | 0.0003520 | 0.01439 | 0.0001346 | 0.05342 |
| regression | | | | | | |
| p-value | | | | | | |
| KM, log-rank | 0.01119 | 0.007909 | 0.001688 | 0.2806 | 0.003837 | 0.1086 |
| p-value | | | | | | |
| KM median | 995 | 915 | 919 | 934 | 934 | 954 |
| months upper | | | | | | |
| 50^{th} percentile | | | | | | |
| CS group | | | | | | |
| KM median | 1765 | 1540 | 2002 | 1314 | 1836 | 1381 |
| months upper | | | | | | |
| 50th percentile | | | | | | |
| CS group | | | | | | |
| KM median | −770 | −625 | −1083 | −380 | −902 | −427 |
| survival | | | | | | |
| difference | | | | | | |
| months | | | | | | |
| N upper 50^{th} | 106 | 76 | 102 | 75 | 102 | 75 |
| percentile | | | | | | |
| CS group | | | | | | |
| N lower 50^{th} | 98 | 73 | 102 | 74 | 102 | 74 |
| percentile | | | | | | |
| CS group | | | | | | |

Table 6. OS Probabilities Associated with Chemical Complementary of CDR3s and the CMV Antigen, IE2.

CS, complementarity score (Methods); KM, Kaplan-Meier; WXS, whole exome sequence; TCR, T-cell receptor; CDR3, complementarity determining region-3.

| | Electrostatic CS | | Combo CS | | Hydrophobicity CS | |
|---|---|---|---|---|---|---|
| | | Primary | | Primary | | Primary |
| | Blood WXS | Tumor WXS | Blood WXS | Tumor WXS | Blood WXS | Tumor WXS |
| | files as the | files as the | files as the | files as the | files as the | files as the |
| | source of the | source of the | source of the | source of the | source of the | source of the |
| | TCR CDR3s | TCR CDR3s | TCR CDR3s | TCR CDR3s | TCR CDR3s | TCR CDR3s |
| Cox-univariate | 1.89E−06 | 0.0008056 | 0.0002286 | 0.01259 | 9.44E−05 | 0.04436 |
| regression | | | | | | |
| p-value | | | | | | |
| KM, log-rank | 0.001929 | 0.0004078 | 0.0001551 | 0.1328 | 0.002631 | 0.2987 |
| p-value | | | | | | |
| KM median | 919 | 830 | 864 | 954 | 919 | 995 |
| months upper | | | | | | |
| 50^{th} percentile | | | | | | |
| CS group | | | | | | |
| KM median | 1696 | 1836 | 2039 | 1330 | 1836 | 1261 |
| months upper | | | | | | |
| 50th percentile | | | | | | |
| CS group | | | | | | |
| KM median | −777 | −1006 | −1175 | −376 | −917 | −266 |
| survival | | | | | | |
| difference | | | | | | |
| (months) | | | | | | |
| N upper 50^{th} | 102 | 75 | 102 | 75 | 102 | 75 |
| percentile | | | | | | |
| CS group | | | | | | |
| N lower 50^{th} | 102 | 74 | 102 | 74 | 102 | 74 |
| percentile | | | | | | |
| CS group | | | | | | |

Table 7. OS Probabilities Associated with Chemical Complementary of CDR3s and the CMV Antigen, UL298.

CS, complementarity score (Methods); KM, Kaplan-Meier; WXS, whole exome sequence; TCR, T-cell receptor; CDR3, complementarity determining region-3.

| | Electrostatic CS | | Combo CS | | Hydrophobicity CS | |
|---|---|---|---|---|---|---|
| | | Primary | | Primary | | Primary |
| | Blood WXS | Tumor WXS | Blood WXS | Tumor WXS | Blood WXS | Tumor WXS |
| | files as the | files as the | files as the | files as the | files as the | files as the |
| | source of the | source of the | source of the | source of the | source of the | source of the |
| | TCR CDR3s | TCR CDR3s | TCR CDR3s | TCR CDR3s | TCR CDR3s | TCR CDR3s |
| Cox-univariate | 5.92E−07 | 0.005973 | 0.0002809 | 0.02605 | 6.16E−05 | 0.05759 |
| regression | | | | | | |
| p-value | | | | | | |
| KM, log-rank | 0.0005307 | 0.0004557 | 0.0002590 | 0.2915 | 0.001163 | 0.4033 |
| p-value | | | | | | |
| KM median | 934 | 864 | 878 | 995 | 919 | 995 |
| months upper | | | | | | |
| 50^{th} percentile | | | | | | |
| CS group | | | | | | |
| KM median | 2039 | 1836 | 2039 | 1261 | 2002 | 1193 |
| months upper | | | | | | |
| 50th percentile | | | | | | |
| CS group | | | | | | |
| KM median | −1105 | −972 | −1161 | −266 | −1083 | −198 |
| survival | | | | | | |
| difference | | | | | | |
| (months) | | | | | | |
| N upper 50^{th} | 111 | 76 | 102 | 75 | 102 | 75 |
| percentile | | | | | | |
| CS group | | | | | | |
| N lower 50^{th} | 93 | 73 | 102 | 74 | 102 | 74 |
| percentile | | | | | | |
| CS group | | | | | | |

### Example 4. Specificity of the CDR3-CMV Antigen Interactions and OS Probability

The approaches above, related to blood-based TCR, anti-viral CDR3s indicated a strong association with a reduced OS probability, we hypothesized that specificity of the chemical complementarity scoring, OS probability assessments is established via the restriction of the assessments to TRB CDR3s and to viral antigen fragments. This was indeed the case, as the upper and lower 50th percentile groups for the TRB CDR3-pp65 antigen fragment CSs were, in two cases, consistent with above results and in one case did not represent an OS probability distinction (FIGS. 3A, 3B and 3C; AA sequences of the three tested, CMV pp65 fragments are in the FIGS. 3A, 3B and 3C legend).

### Example 5. A Multivariate Analysis of the Anti-Viral CMV Survival Associations

To determine whether the OS distinction represented by the upper and lower 50th percentiles for the Combo CS groups, for the blood TCR CDR3-CMV UL40 antigen pairs (Table 3), represented significance when accounting for other common NBL clinical variables, a multivariate was performed, Cox regression analysis using variables that all represented statistical significance in a univariate analysis. Results indicated that the OS distinction based on the Combo CS groups maintained its significance in the multivariate analysis (Table 8).

### Table 8. Multivariate Analysis of the Combo CS for TCR CDR3-CMV UL40 Antigen (Table 3) Complementarity and Various Clinical Features.

| | | | Odds ratios |
|---|---|---|---|
| | | | for the 95% |
| | | | confidence |
| Variable category | B-factor | Significance | interval |
| Combo CS for TCR | 0.455 | 0.046 | 1.007-2.468 |
| CDR3-CMV | | | |
| Age at diagnosis | −0.270 | 0.279 | 0.468-1.245 |
| MYCN amplification | 0.127 | 0.637 | 0.669-1.927 |
| Tumor ploidy | −0.086 | 0.702 | 0.592-1.423 |
| Mitosis-karyorrhexis | 0.149 | 0.564 | 0.700-1.924 |
| index | | | |

### Methods

Isolation of TCR recombination reads from Therapeutically Applicable Research to Generate Effective Treatments (TARGET), NBL exome (WXS) files. The process for mining the immune receptor recombination reads from the WXS files has been extensively. The latest version of the software used for this IR recombination read extraction process is at github.com, 2021. A complete collection of data for the recovery of the TRA and TRB recombination reads is in supporting online material (SOM). The TARGET WXS files, representing database of genotypes and phenotypes (dbGaP) designation, phs000218, were accessed according to dbGaP protocol approval number 16405.

Identifying anti-viral CDR3s in the TCR NBL dataset. The TRA and TRB anti-viral CDR3 dataset was downloaded from VDJdb (vdjdb.cdr3.net) and used to match to NBL TRA and TRB CDR3s in the NBL dataset obtained as indicated above.

Survival analysis. To assess differences in overall survival (OS) probabilities, we applied the Kaplan-Meier (KM) log-rank test. KM curves were generated using R v4.2.2 with CRAN packages ‘survminer’ v0.4.9 and ‘survival’ v3.4.0. Nominal log-rank p-values less than 0.05 were considered statistically significant. Base R code is available at github.com/MJD-BlanckGroup/Survival (Terry M. Therneau, Patricia M. Grambsch (2000).

Data: Extending the Cox Model. The OS analyses were confirmed by use of the cbioportal.org web tool.

Multivariate analysis. The multivariate analysis was conducted with the statistics package for the social sciences (IBM).

### Discussion

The above data indicated that the presence of virus, or higher levels of virus, in a subset of NBL patients. Regardless, the above approach, applied previously to adult cancers but now for the first time to childhood cancer, strongly supports the possibility that the presence of blood-based anti-viral CDR3s is associated with a worse outcome. This result was specifically and most cogently established for CMV, previously associated with NBL. Notably, the anti-CMV CDR3 identifications were not limited to specific CDR3 AA sequences established by previous studies. If that had been the case, there could have been the possibility that cases lacking anti-CMV CDR3s simply had anti-CMV CDR3s with as yet unknown AA sequences. However, the chemical complementarity scoring algorithm applied independently of any previous anti-CMV CDR3 AA sequence identifications strongly supported the basic result: anti-CMV CDR3s associated with a worse outcome (Tables 3-7).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred examples of the invention and that such changes and modifications can be made without departing from the spirit of the invention. It is, therefore, intended that the appended claims cover all such equivalent variations as fall within the true spirit and scope of the invention.

SEQUENCES

SEQ ID NO: 1 (CMV pp65 peptide fragment-1)
MESRGRRCPEMISVLGPISGHVLKAVFSRGDTPVLPHETRLLQTGIH
SEQ ID NO: 2 (CMV pp65 peptide fragment-2)
AVIHASGKQMWQARLTVSGLAWTRQQNQWKEPDVYYTS
SEQ ID NO: 3 (CMV pp65 peptide fragment-3)
AIRETVELRQYDPVAALFFFDIDLLLQRGPQYS

What is claimed is:

1. A method of determining overall survival in a subject with neuroblastoma, comprising:
a) obtaining a blood sample from the subject, wherein the blood sample comprises a T-cell receptor (TCR);
b) extracting complementarity determining region 3 (CDR3) amino acid (AA) sequences from the T-cell receptor (TCR) in the blood sample, thereby obtaining extracted CDR3 AA sequences;
c) identifying an exact match of extracted CDR3 AA sequences to known anti-viral CDR3 AA sequences from the blood sample; thereby obtaining an exact match anti-viral CDR3 AA sequence; and
d) correlating presence of the exact match anti-viral CDR3 AA sequence with overall survival of the subject,
wherein the presence of the exact match anti-viral CDR3 AA sequence in the blood sample is correlated to poor overall survival compared to a reference control, wherein the reference control does not have the exact match anti-viral CDR3 AA sequence in the blood sample.

2. The method of claim 1, wherein the known anti-viral CDR3 AA sequences of one or more viruses selected from the group consisting of Cytomegalovirus (CMV), Epstein-Barr virus (EBV), Hepatitis C virus (HCV), human immunodeficiency virus (HIV), Influenza A, and SARS-CoV-2.

3. The method of claim 1, wherein the T cell receptor (TCR) comprises an alpha chain or a beta chain.

4. A method of determining overall survival in a subject with neuroblastoma, comprising:
a) obtaining a sample from the subject, wherein the sample comprises a T-cell receptor (TCR);
b) extracting complementarity determining region 3 (CDR3) from the T-cell receptor (TCR) in the sample, thereby obtaining extracted CDR3;
c) obtaining a chemical complementarity score (CS) by interacting known viral antigens to the extracted CDR3 from the sample;
d) calculating the CS; and
e) correlating the CS with overall survival of the subject,
wherein a high CS is correlated to poor overall survival compared to a reference control, wherein the reference control has low CS.

5. The method of claim 4, wherein the high CS comprises a high chemical complementarity between the known viral antigens and the extracted CDR3.

6. The method of claim 4, wherein the CS is calculated using hydrophobic interactions, electrostatic interactions, or a combination thereof.

7. The method of claim 4, wherein the sample comprises blood or tumor biopsy.

8. The method of claim 4, wherein the T cell receptor (TCR) comprises an alpha chain or a beta chain.

9. The method of claim 4, wherein the known viral antigens of one or more viruses selected from the group consisting of Cytomegalovirus (CMV), Epstein-Barr virus (EBV), Hepatitis C virus (HCV), human immunodeficiency virus (HIV), Influenza A, and SARS-CoV-2.

10. A method of treating neuroblastoma in a subject, comprising:
a) obtaining a sample from the subject, wherein the sample comprises a T-cell receptor (TCR);
b) extracting complementarity determining region 3 (CDR3) amino acid (AA) sequences from the T-cell receptor (TCR) in the sample, thereby obtaining extracted CDR3 AA sequences;
c) identifying an exact match of extracted CDR3 AA sequences to anti-Cytomegalovirus (CMV) CDR3 AA sequences from the sample; thereby obtaining an exact match anti-CMV CDR3 AA sequence;
d) correlating presence of the exact match anti-CMV CDR3 AA sequence with overall survival of the subject; and
e) administering a therapeutically effective amount of an anti-CMV comprising Ganciclovir or Valganciclovir to the subject with a presence of the exact match anti-CMV CDR3 sequence,
wherein the presence of the exact match anti-CMV CDR3 AA sequence in the sample is correlated to poor overall survival.

11. The method of claim 10, wherein the sample comprises blood or tumor biopsy.

12. The method of claim 10, wherein the T cell receptor (TCR) comprises an alpha chain or a beta chain.

13. A method of treating neuroblastoma in a subject, comprising:
a) obtaining a sample from the subject, wherein the sample comprises a T-cell receptor (TCR);
b) extracting complementarity determining region 3 (CDR3) from the T-cell receptor (TCR) in the sample, thereby obtaining extracted CDR3;
c) obtaining a chemical complementarity score (CS) by interacting CMV antigen to the extracted CDR3 from the sample;
d) calculating the CS;
e) correlating the CS with overall survival of the subject; and
f) administering a therapeutically effective amount of an anti-CMV comprising Ganciclovir or Valganciclovir to the subject with a high CS, wherein a high CS is correlated to poor overall survival.

14. The method of claim 13, wherein the high CS comprises a high chemical complementarity between the known viral antigens and the extracted CDR3.

15. The method of claim 13, wherein the CS is calculated using hydrophobic interactions, electrostatic interactions, or a combination thereof.

16. The method of claim 13, wherein the sample comprises blood or tumor biopsy.

17. The method of claim 13, wherein the T cell receptor (TCR) comprises an alpha chain or a beta chain.

18. A method of treating neuroblastoma in a subject based on correlation between chemical complementary scoring and gene amplification, comprising:
a) obtaining a sample from the subject, wherein the sample comprises a T-cell receptor (TCR);
b) extracting complementarity determining region 3 (CDR3) from the T-cell receptor (TCR) in the sample, thereby obtaining extracted CDR3;
c) obtaining a chemical complementarity score (CS) by interacting known viral antigen to the extracted CDR3 from the sample;
d) calculating the CS;
e) correlating the CS with overall survival of the subject; wherein a high CS is correlated to poor overall survival compared to a reference control, wherein the reference control has low CS;
f) determining gene amplification status of MYCN gene in the sample;
g) classifying the subject into a high-risk group based on the high CS and MYCN gene amplification in the sample; and
h) administering a therapeutically effective amount of an anti-viral comprising Ganciclovir or Valganciclovir to the high-risk group.

19. The method of claim 18, wherein the T cell receptor (TCR) comprises an alpha chain or a beta chain.

20. The method of claim 18, wherein the high CS comprises a high chemical complementarity between the known viral antigens and the extracted CDR3.

21. The method of claim 18, wherein the CS is calculated using hydrophobic interactions, electrostatic interactions, or a combination thereof.

22. The method of claim 18, wherein the sample comprises blood or tumor biopsy.

23. The method of claim 18, wherein the known viral antigens of one or more viruses selected from the group consisting of Cytomegalovirus (CMV), Epstein-Barr virus (EBV), Hepatitis C virus (HCV), human immunodeficiency virus (HIV), Influenza A, and SARS-CoV-2.

24. A method of treating neuroblastoma in a subject based on correlation between exact match sequencing and gene amplification, comprising:
a) obtaining a sample from the subject, wherein the sample comprises a T-cell receptor (TCR);
b) extracting complementarity determining region 3 (CDR3) amino acid (AA) sequences from the T-cell receptor (TCR) in the sample, thereby obtaining extracted CDR3 AA sequences;
c) identifying an exact match of extracted CDR3 AA sequences to known anti-viral CDR3 AA sequences from the sample; thereby obtaining an exact match anti-viral CDR3 AA sequence;
d) correlating presence of the exact match anti-viral CDR3 AA sequence with overall survival of the subject, wherein the presence of the exact match anti-viral CDR3 AA sequence in the sample is correlated to poor overall survival;
e) determining gene amplification status of MYCN gene in the sample;
f) classifying the subject into a high-risk group based on the presence of the exact match anti-viral CDR3 AA sequence and MYCN gene amplification in the sample; and
g) administering a therapeutically effective amount of an anti-viral comprising Ganciclovir or Valganciclovir to the high-risk group.

25. The method of claim 24, wherein the known anti-viral CDR3 AA sequences of one or more viruses selected from the group consisting of Cytomegalovirus (CMV), Epstein-Barr virus (EBV), Hepatitis C virus (HCV), human immunodeficiency virus (HIV), Influenza A, and SARS-CoV-2.

26. The method of claim 24, wherein the sample comprises blood or tumor biopsy.
